(19) United States
(12) Patent Application Publication
(10) Pub. No.: US 2024/0185994 A1
(43) Pub. Date: Jun. 6, 2024

(54) METHOD AND SYSTEM FOR PROVIDING CERTIFICATION OF VACCINE INOCULATION AND POST-INOCULATION MANAGEMENT

(71) Applicant: BLOCKCHAIN LABS INC., Seoul (KR)

(72) Inventors: Yong Tae KIM, Seoul (KR); Jee Yong UM, Seoul (KR); Byung Wan LIM, Seoul (KR)

(73) Assignee: BLOCKCHAIN LABS INC., Seoul (KR)

(21) Appl. No.: 18/550,263

(22) PCT Filed: Mar. 22, 2021
(86) PCT No.: PCT/KR2021/003521

§ 371 Date: Sep. 12, 2023

(30) Foreign Application Priority Data
Mar. 15, 2021 (KR) 10-2021-0033413

(51) Int. Cl. G16H 40/20; G16H 50/80; H04L 9/00; H04L 9/32

(52) U.S. Cl. CPC G16H 40/20; G16H 50/80; H04L 9/3247; H04L 9/50

(57) ABSTRACT

The present disclosure relates to a method for vaccination management including at least: transmitting, by a medical institution device, a vaccination certificate issuance request, an identity certification verifiable credential (VC) for a user, a vaccination agency VC for a medical institution, and a digital signature of the medical institution, to a trusted institution server; verifying, by the trusted institution server, the vaccination agency VC based on a digital signature of a VC issuer included in the vaccination agency VC and the identifier of the trusted institution stored in a public distributed ledger; determining, by the trusted institution server, whether there is an authority for the vaccination certificate issuance request based on the vaccination agency VC and a medical institution database stored in the trusted institution server; and issuing, by the trusted institution server, a vaccination certification VC.

### TECHNICAL FIELD

Embodiments disclosed herein relate to technology for issuing and distributing medical certificates and technology for follow-up management.

### BACKGROUND ART

When a virus spreads around the world, the infectious disease caused by this virus leads to a global pandemic. A vaccine is developed to solve the pandemic, and vaccination begins when the vaccine is completed. In this case, people are divided into vaccinated persons and unvaccinated persons, and in order to distinguish the vaccinated persons from the unvaccinated persons, a certificating means capable of certificating the vaccinated person is required.

Further, when vaccination is performed with a newly developed vaccine, information on side effects of the vaccine or information on symptoms after vaccination may be continuously updated. Therefore, it is necessary to provide follow-up management for a vaccinated person.

### DISCLOSURE OF THE INVENTION

### Technical Problem

The distinction between vaccinated and unvaccinated persons becomes essential, especially for highly contagious diseases. Due to its high contagiousness, certification of being the vaccinated person may be required, for example, to have authority to access a place where many people may come into contact. A certificate may be issued to certify that a person has been vaccinated. The certificate may include information about a name of the vaccinated person, vaccine information, a medical institution, and the like.

When the certificate is a paper certificate, it is difficult to check the authenticity of the certificate itself and the risk of leakage of personal information is high.

Current electronic certificates are issued only by specific trusted institutions. A procedure for issuing an electronic certificate includes a user certification procedure and a payment procedure, which makes the procedure for issuing complicated, and thus the procedure is not convenient for users. Even if issuance is completed through the complicated procedure for issuing, since the certificate is distributed in the form of printed paper (submitted to an institution, or the like), it is difficult to reuse it and there is the inconvenience of having to repeat the procedures for issuing and printing again.

Various embodiments disclosed herein are directed to providing a method and system capable of simplifying certificate issuance and distribution procedures and allowing users to conveniently certificate a status of vaccination. In addition, it is intended to prevent the risk of leakage of personal information in a certificating process and allow an easy certification between individuals. It is intended to enable certification around the world by issuing a certificate once. In addition, it is intended to provide feedback reflecting medical information updated in real time to hospitals or vaccinated persons, and to provide a follow-up management after vaccination.

### Technical Solution

A method for vaccination management according to one embodiment disclosed herein includes a trusted institution server associated with a trusted institution, a medical institution device associated with a medical institution, a user device associated with a user, and a public distributed ledger, a first storage space of the public distributed ledger being set to be modifiable only by the trusted institution server, an identifier of the trusted institution and an identifier of the medical institution being stored in the first storage space, the method including transmitting, by the medical institution device, a vaccination certificate issuance request including vaccination information, an identity certification verifiable credential (VC) for the user received from the user device, a vaccination agency VC for the medical institution, and a digital signature of the medical institution, to the trusted institution server, verifying, by the trusted institution server, the vaccination agency VC based on a digital signature of a VC issuer included in the vaccination agency VC and the identifier of the trusted institution stored in the public distributed ledger, in response to the vaccination certificate issuance request, determining, by the trusted institution server, whether or not there is an authority for the vaccination certificate issuance request based on the vaccination agency VC and a medical institution database stored in the trusted institution server, in response to the vaccination certificate issuance request, issuing, by the trusted institution server, a vaccination certification VC including the vaccination information, at least some data included in the identity certification VC, and a digital signature of the trusted institution, based on the verification result and the determination result, and transmitting, by the trusted institution server, the vaccination certification VC to the user device.

### Advantageous Effects

According to the embodiments disclosed herein, certification of the status of vaccination can be made while the exposure of personal information is minimized. In addition, certification with high reliability can be made between individuals without a national institution or trusted institution as an intermediary. Besides, various effects may be provided that are directly or indirectly identified through the present disclosure.

### BRIEF DESCRIPTION OF THE DRAWINGS

FIG. 1 is a schematic diagram of a system for issuing a vaccination digital certificate and verifying whether vaccination has been performed according to one embodiment.

FIG. 2 is a pair of block diagrams of an identity authentication server and a vaccination management server according to one embodiment.

FIG. 3 is a pair of block diagrams of a medical institution electronic device and a personal electronic device according to one embodiment.

FIG. 4 is a signal flow diagram for a method in which the identity authentication server issues an identity certificate to the personal electronic device according to one embodiment.

FIG. 5 is a signal flow diagram for a method in which the vaccination management server issues a vaccination agency certificate to the medical institution electronic device according to one embodiment.

FIG. 6 is a signal flow diagram for a method of registering a vaccination practitioner in the vaccination management server according to one embodiment.

FIG. 7a is a signal flow diagram for a method of issuing a vaccination certificate according to one embodiment.

FIG. 7b is an example of a screen interface displayed on the medical institution electronic device according to one embodiment.

FIG. 7c is an example of a screen interface displayed on the personal electronic device according to one embodiment.

FIG. 8 is a signal flow diagram for a method of certificating a status of vaccination according to one embodiment.

FIG. 9a is an example of a screen interface showing a certification process between an A electronic device and a B electronic device according to one embodiment.

FIG. 9b is an example of a screen interface for selecting information to be included in a certificate processed in the A electronic device according to one embodiment.

FIG. 10a is a signal flow diagram of a follow-up management service according to one embodiment is illustrated.

FIG. 10b is an example of a screen interface in a follow-up management process according to one embodiment is illustrated.

With respect to the description of the drawings, the same or similar reference signs may be used for the same or similar elements.

### MODE FOR CARRYING OUT THE INVENTION

Hereinafter, various embodiments of the present disclosure will be described with reference to the accompanying drawings. However, this is not intended to limit the present disclosure to specific embodiments, and should be understood to include various modifications, equivalents, and/or alternatives of the embodiment of the present disclosure.

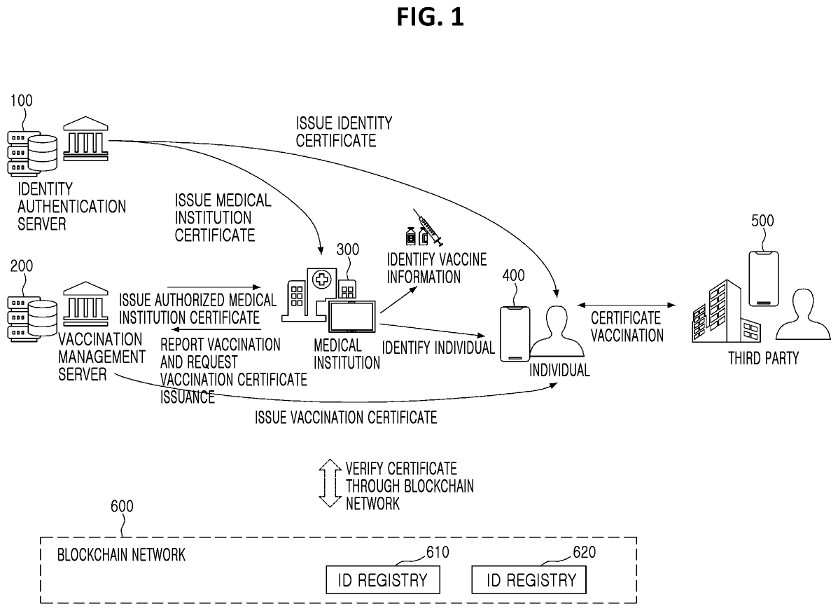

FIG. 1 is a schematic diagram of a system 10 for issuing a vaccination digital certificate and verifying whether vaccination has been performed according to one embodiment (hereinafter, and a system 10). An issuance verification process for the vaccination digital certificate performed on the system 10 will be described as follows.

A vaccination certificate (hereinafter, a vaccination certificate) that certificates a status of vaccination may be issued in the form of a digital certificate. The vaccination certificate may be a data set including data associated with vaccination. The data may include sensitive information about an individual related to vaccination.

A vaccination management agency may be an institution or organization that manages matters related to vaccination. The vaccination management agency may monitor a series of processes of importing, distributing, and inoculating vaccines, and store and manage records thereof. The vaccination management agency is an institution run by a state and may be understood as a trusted institution.

An identity certificate may authority be an institution or organization that manages identity information about individuals and medical institutions. An identity certificate authority may manage identity information about an individual that enables identification of a specific individual. The identity certificate authority may issue a certificate (hereinafter referred to as an identity certificate) for at least one item of identity information. The identity certificate authority may manage basic information about a medical institution that enables identification of a specific medical institution. The identity certificate authority may issue a certificate (hereinafter referred to as a medical institution certificate) for at least one item of the basic information about a medical institution. The identity certificate authority may perform certification of medical institutions and identity of a vaccinated person, which are necessary for vaccination. The identity certificate authority is an institution run by a state and may be understood as a trusted institution.

Vaccination may be performed by a medical institution certified to administer vaccines. The vaccination management agency may select a medical institution capable of performing vaccination among medical institutions or manage a list of previously selected medical institutions. The vaccination management agency may check basic information about a vaccination agency and issue vaccination agency certificates.

In order to issue vaccination certificates, the identity certificate authority, the vaccination management agency, and the medical institution may cooperate with each other. Vaccination certificates may be issued by the vaccination management agency. The identity certificate authority may provide the vaccination management agency with basic information about the medical institution for issuing the vaccination certificate.

In various embodiments, the vaccination certificate may be issued directly by the medical institution. For example, in countries where there is no national institution such as the identity certificate authority and the vaccination management agency, the vaccination certificate may be issued directly by the medical institution. In this case, both the role of the identity certificate authority and the role of the vaccination management agency may be performed by the medical institution.

Referring to FIG. 1, the system 10 may include an identity authentication server 100, a vaccination management server 200, a medical institution electronic device 300, a personal electronic device 400, and a third party electronic device 500. Although the identity authentication server 100 and the vaccination management server 200 may include a plurality of authentication servers, the system 10 including one authentication server will be disclosed.

In various embodiments, the identity certificate authority and the vaccination management agency may be integrated. For example, one trusted institution may serve as the identity certificate authority and the vaccination management agency. Hereinafter, the identity authentication server 100 operated by the identity certificate authority and the vaccination management server 200 operated by the vaccination management agency are disclosed, but the operations of each server may be implemented as a module of one server. Alternatively, the roles of the identity certificate authority and the vaccination management agency may be performed by a plurality of trusted institutions in a divided manner. Accordingly, the trusted institution server may be operated by at least one trusted institution. The trusted institution server may include a plurality of servers (the identity authentication server 100 and the vaccination management server 200).

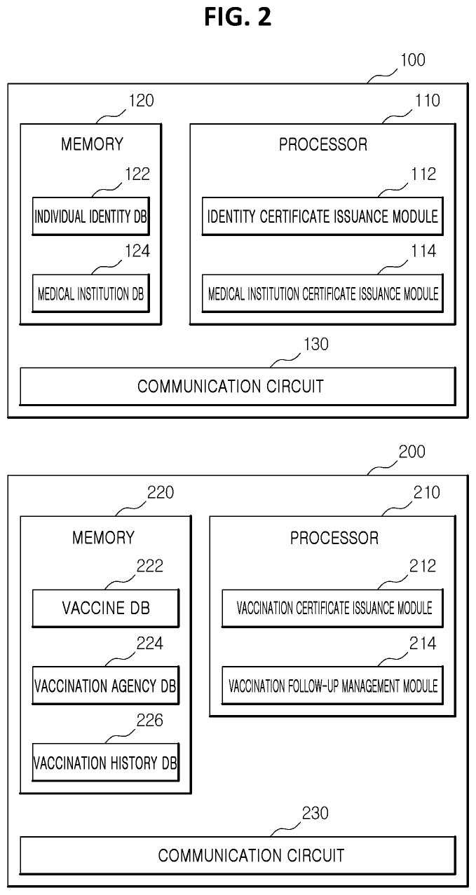

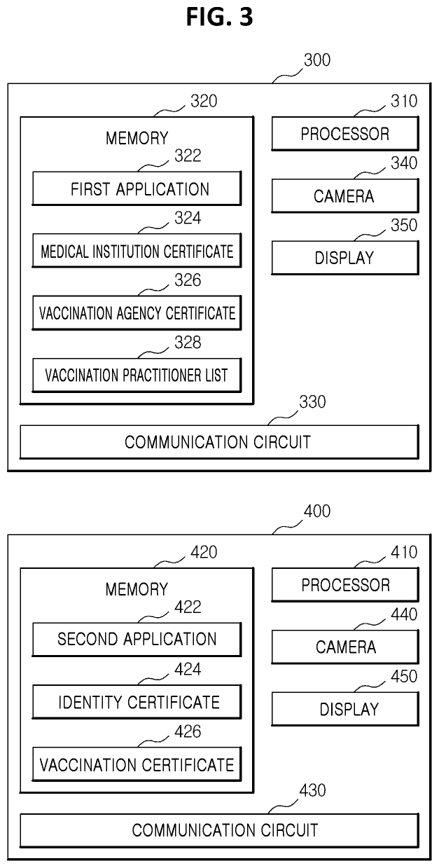

The identity authentication server 100 may be a server device operated by the identity certificate authority. The vaccination management server 200 may be a server device operated by the vaccination management agency. The medical institution electronic device 300 may be an electronic device (e.g., a PC, a tablet, or a server device) possessed by the medical institution that performs vaccination. The personal electronic device 400 may be an electronic device (e.g., a smartphone, a tablet, or a personal PC) of an individual to be vaccinated. The third party electronic device 500 may be an electronic device (e.g., a PC, a tablet, or a server device) owned by another person, company, or the like, that checks a status of vaccination for a specific individual. The structure and operation of the server device and the electronic device will be described below with reference to FIGS. 2 and 3.

The identity authentication server 100 may issue an identity certificate for an individual and transmit the issued identity certificate to the personal electronic device 400. The identity authentication server 100 may issue a medical institution certificate for a medical institution, and transmit the issued certificate to the medical institution electronic device 300. The personal electronic device 400 may store the received identity certificate. The medical institution electronic device 300 may store the received medical institution certificate.

In various embodiments, the medical institution certificate may be understood as a confirmation that allows the medical institution to be identified as a specific medical institution. The medical institution certificate may be a confirmation for the medical institution itself or an identity certificate for a representative of the medical institution.

The vaccination management server 200 may issue a vaccination agency certificate for the specific medical institution and transmit the issued certificate to the medical institution electronic device 300. The medical institution electronic device 300 store the received vaccination agency certificate.

When the vaccination management server 200 issues the vaccination agency certificate, the vaccination management server 200 may receive the medical institution certificate from the identity authentication server 100 or the medical institution electronic device 300. The vaccination management server 200 may identify whether the medical institution is the specific medical institution having vaccination authority based on the medical institution certificate, and then issue the vaccination agency certificate.

The medical institution electronic device 300 may identify vaccine information. Vaccine information may include information about a vaccine to be administered to a person to be vaccinated. Vaccine information may include, for example, a name of a vaccine, a manufacturer, clinical information, identification information, and distribution information. For example, the medical institution electronic device 300 may receive the vaccine information by recognizing a code (e.g., a bar code or a QR code) attached to a vaccine. Alternatively, the medical institution electronic device 300 may receive a direct input of vaccine information from a medical person.

The medical institution electronic device 300 may identify an individual who is a vaccinated person. For example, the medical institution electronic device 300 may receive an identity certificate stored in the personal electronic device 300 and identify an individual based on the received identity certificate. After vaccination is completed, the medical institution electronic device 300 may transmit the identified vaccine information, the identified personal information, and the vaccination information to the vaccination management server 200. The vaccination information may include information necessary for reporting the inoculation fact, such as a vaccination time, a vaccination location, and a status of the vaccinated person.

The vaccination management server 200 may issue a vaccination certificate based on the received vaccine information, personal information, and vaccination information. The vaccination management server 200 may transmit the issued vaccination certificate to the personal electronic device 400.

The personal electronic device 300 may store the vaccination certificate. The personal electronic device 300 may transmit at least some information included in the vaccination certificate to the third party electronic device 500 owned by another subject requesting vaccination certification.

The certificate disclosed herein may be digitally signed by the issuer. The digital signature may be a means of proving the identity of the issuer. For example, the issuer may digitally sign a certificate using an identifier corresponding to the digital identity of the issuer. A recipient of the certificate may check who the issuer of the certificate is through the digital signature. Each subject (the identity authentication server 100, the vaccination management server 200, the medical institution electronic device 300, the personal electronic device 400) included in the system 10 may have its own identifier, and use the identifier to issue a digitally signed certificate.

In various embodiments, the digital signature may be implemented by various algorithms. A key generation algorithm, a signing algorithm, and a signature verifying algorithm may be utilized. In one example, the RSA algorithm may be utilized.

The identity authentication server 100 may digitally sign the identity certificate and the medical institution certificate with its own identifier. The vaccination management server 200 may digitally sign the vaccination certificate with its own identifier. The recipient who receives the identity certificate or the vaccination certificate may check the issuer through the digital signature, and may trust the content of the certificate.

For example, in the case of the key generation algorithm, the identity authentication server 100 or the vaccination management server 200 may sign the certificate with its private key. The certificate recipient may check the owner of the digital signature with a public key derived from the digital signature of the identity authentication server 100 or the vaccination management server 200.

The certificate disclosed herein may be designed to be verifiable by a third party. That is, certificates issued and distributed on the system 10 may be verified by all other than a specific trusted institution (e.g., an identity certificate authority or a vaccination management agency). For example, in the case of the key generation algorithm, the public key may not be stored in a specific centralized central institution, but may be stored in a decentralized peer-to-peer (P2P) network such as a federated ID management system or a distributed ledger of a plurality of trusted institutions. Thereby, the certificate may be verified by using an identifier (e.g., a public key) value stored in a repository that may be accessible to all.

In one embodiment, when the repository is managed by a trusted institution such as the identity certificate authority and the vaccination management agency, that is, when only the trusted institution has the authority to input, modify, or delete data in a specific repository, reliable data that is accessible to all may be stored in the repository (e.g., a first ID registry 610).

In various embodiments, the personal electronic device 400 may generate a certificate by processing some of the content of the vaccination certificate. The personal electronic device 400 may transmit the processed certificate to the third party electronic device 500. The purpose of processing is to protect privacy by not transmitting all the contents of the vaccination certificate to a third party and ensuring that only essential contents are included. For example, when it is necessary to verify only the fact of the status of vaccination, a certificate containing only “the status of vaccination” may be transmitted to the third party electronic device 500 excluding information such as “what kind of vaccine was administered” and “in which medical institution the vaccination was performed”.

In one embodiment, the system 10 may be operated based on a blockchain network 600. The blockchain network 600 of the system 10 may include at least one blockchain network among known public blockchains. Key values of the identifiers of respective subjects 100, 200, 300, and 400 may be stored in the blockchain network 600. For example, the identifier of each of the subjects 100, 200, 300, and 400 may be an account or address value on the blockchain network 600.

By utilizing the blockchain network 600, the system 10 may function as a global system. Since certificates may be issued and verified based on the blockchain network 600, which is a global platform, the vaccination certificate may be issued and verified without being restricted by trusted institutions in each country or different certification systems in each country. Hereinafter, the system 10 based on the blockchain network 600 will be described as an example. However, the system 10 is not limited thereto, and other open and reliable networks may be employed.

In one embodiment, the identity authentication server 100, the vaccination management server 200, the medical institution electronic device 300, and the personal electronic device 400 may communicate with the blockchain network 600 and have at least one application that allows access to accounts on the blockchain network 600 stored. The at least one application may include a wallet application of the blockchain network 600.

In one embodiment, a certificate issued and distributed by the system 10 may have a verifiable credential (VC) specification designed according to the W3C standard protocol. Each of the subjects 100, 200, 300, and 400 of the system 100 may be an issuer of VC. The VC may include identifier information about the issuer and the issuer's digital signature. The VC may include owner identifier information for the VC. For example, when the issuer of VC issues the VC, the issuer may issue the VC including identifier information about a target subject to which the VC is to be issued, that is, an owner.

For example, the processed certificate may have a specification of verifiable presentation (VP) designed according to the W3C standard protocol. The VP may be constituted by some claims among claims included in one VC, or may be constituted by a plurality of VCs. Further, the VP may include a plurality of different VCs issued by different issuers. The subjects 100, 200, 300, and 400 of the system 10 may create or issue a VP including only necessary information from one or more VCs. In this case, the VP may include both a digital signature of a creator and a digital signature of the issuer of the VC included in the VP.

Digital signatures or digital identifiers used under the blockchain network 600 may be understood as decentralized Identifiers (DIDs) according to the W3C standard protocol. Digital signatures may be understood as being created based on the DID.

In one embodiment, the blockchain network 600 may store ID registries 610 and 620. The ID registries 610 and 620 may store identifiers (e.g., DIDs) of subjects of the system 10. For example, identifiers (e.g., the DIDs) of the identity authentication server 100, the vaccination management server 200, the medical institution electronic device 300, and the personal electronic device 400 may be stored in the ID registries 610 and 620.

Each of the subjects 100, 200, 300, and 400 of the system 10 may access the ID registries 610 and 620 of the blockchain network 600 to mutually verify certificates, and view the identifiers stored in the registries.

In one embodiment, the blockchain network 600 may include the first ID registry 610 and the second ID registry 620. The first ID registry 610 may be understood as a storage space managed by the identity authentication server 100 and the vaccination management server 200. Data stored in the first ID registry 610 may be input, modified, or deleted by the trusted institutions 100 and 200. Therefore, the medical institution electronic device 300 and the personal electronic device 400 may view the data stored in the first ID registry 610, but may not modify or delete the data stored in the first ID registry 610 or may not add new data to the first ID registry 610.

Identifiers of the identity certificate authority and the vaccination management agency, which are trusted institutions of the system 10, may be stored in the first ID registry 610. Any subject may check the authenticity of the certificate issued by the trusted institution by viewing the first ID registry 610. The certificate issued by the trusted institution may include a digital signature of the trusted institution, and the subject receiving the certificate may verify the digital signature based on an identifier stored in the first ID registry 610. In addition, since the first ID registry 610 is managed only by the trusted institution, the subject viewing data stored in the first ID registry 610 may always trust the authenticity of the data.

The data stored in the second ID registry 620 may be input, modified, or deleted by each of the subjects 100, 200, 300, and 400. Therefore, information provided by the trusted institutions 100 and 200 may be stored in the blockchain network 600, and the subject viewing the information may trust the information.

In various embodiments, the VC may include identifier information about the issuer (e.g., the DID or public key) and identifier of an issuance target (e.g., the DID or public key). For example, when an identity certification VC including identity information about the owner/user of the personal electronic device 400 is issued by the identity authentication server 100, the VC may include identifier information (a first identifier) about the identity certificate authority and identifier information (a second identifier) about the individual user. This may include meaning that the VC is issued by the identity certificate authority, which is the owner of the first identifier, and issued to the user, which is the owner of the second identifier.

For example, the personal electronic device 400 may create an identity certification VP by signing an identity certification VC with the second identifier of the user. The identity certification VP includes a digital signature of the identity certificate authority that is the issuer of the VC, a digital signature of the user, and an identifier information (DID) about the user. The personal electronic device 400 may submit the identity certification VC to the vaccination management agency by transmitting the identity certification VP to the vaccination management server 200. Hereinafter, an operation in which a VP for one VC is submitted may be abbreviated as a VC being submitted.

The vaccination management server 200 may perform verification on the identity certification VP. In various embodiments, verification of VC and VP may be understood as verification of the digital signature included in the VP.

In the above example, the vaccination management server 200 may verify the digital signature of the identity certificate authority that is the VC issuer. The vaccination management server 200 may compare an identifier derived from the digital signature of the identity certificate authority with an identifier of the identity certificate authority stored in the trusted first ID registry 610. When the two match, the fact that the identity certification VC is issued by the trusted institution having legitimate authority to issue the identity certificate may be proved. Hereinafter, verification of the digital signature of the issuer of the VC may be performed based on the identifier stored in the first ID registry 610 of the blockchain network 600.

Furthermore, in the above example, the vaccination management server 200 may verify the digital signature of the user, which is the creator of the VP. The vaccination management server 200 may compare the identifier derived from the digital signature of the user with an identifier described as the issuance target in the identity certification VC. When the two match, the vaccination management server 200 may prove that the user who currently owns the identity certification VC is the legitimate owner of the identity certification VC. Hereinafter, verification of the VP created for at least some information included in the VC may be performed based on an identifier included in the VC.

FIG. 2 is a pair of block diagrams of the identity authentication server 100 and the vaccination management server 200 according to one embodiment. In various embodiments, the identity authentication server 100 and the vaccination management server 200 may be integrated. The identity authentication server 100 and the vaccination management server 200 are illustrated as one server device, but may be a plurality of server devices.

In one embodiment, the identity authentication server 100 may include a processor 110, a memory 120, and a communication circuit 130. The processor 110 may control the overall operation of the identity authentication server 100. The identity authentication server 100 may transmit and receive data to and from the other subjects 200, 300, and 400 of the system 10 through the communication circuit 130.

In one embodiment, the memory 120 may include a personal identity database (DB) 122 containing identity information about individuals to whom identity certificates are to be issued. The personal identity DB 122 may include types of data shown in Table 1. An identity certificate for an individual may be issued based on the personal identity DB 122.

TABLE 1

| | |
|---|---|
| 1 | Identifier (e.g., DID) |
| 2 | Name |
| 3 | Date of birth |
| 4 | Phone number |
| 5 | Personal identification number |
| | (e.g., resident registration number) |
| 6 | Nationality |
| 7 | Push token |

The memory 120 may include a medical institution DB 124 for medical institutions to which the medical institution certificates are to be issued. The medical institution DB 124 may include types of data shown in Table 2. A medical institution certificate for a medical institution may be issued based on the medical institution DB 124.

TABLE 2

| | |
|---|---|
| 1 | Medical institution identification number |
| | (e.g., institution registration number) |
| 2 | Name of medical institution |
| 3 | Address of medical institution |
| 4 | Representative name |
| 5 | Representative phone number |
| 6 | Medical institution identifier (e.g., DID) |

In one embodiment, the processor 110 may include an identity certificate issuance module 112 and a medical institution certificate issuance module 114. The processor 110 may drive the identity certificate issuance module 112 and the medical institution certificate issuance module 114 by executing instructions stored in the memory 120. Operations performed by the identity certificate issuance module 112 and the medical institution certificate issuance module 114 may be understood as operations performed by the processor 110. Other operations described as being performed by the identity authentication server 100 may be understood as operations performed by the processor 110. The identity certificate issuance module 112 may issue an identity certificate using the personal identity DB 122 stored in the memory 120. For example, the identity certificate issuance module 112 may generate an identity certificate including at least a part of data included in the personal identity DB 122. The generated identity certificate may be digitally signed with an identifier of the identity authentication server 100. The identity certificate may include an identifier of an individual to whom is the identity certificate is to be issued.

The medical institution certificate issuance module 114 may issue a medical institution certificate using the medical institution DB 124 stored in the memory 120. For example, the identity certificate issuance module 112 may generate a medical institution certificate including at least a part of data included in the medical institution DB 124. The generated medical institution certificate may be digitally signed with the identifier of the identity authentication server 100. The medical institution certificate may include an identifier of a medical institution to whom is the medical institution certificate is to be issued.

In one embodiment, the identity authentication server 100 may include a wallet for the identity authentication server 100 to communicate with the blockchain network 600. The wallet may include an account of the identity authentication server 100 on the blockchain network 600. For example, the identity authentication server 100 may store a private key in the memory 120. When the identity authentication server 100 issues a certificate, the identity authentication server 100 may perform signing with its own private key.

In various embodiments, the wallet may include a DID agent. The DID agent may receive a DID on the blockchain network 600, retrieve the DID, and change the DID. In various embodiments, the wallet may include the DID agent. The DID agent may receive a DID on the blockchain network 600, retrieve the DID, and change the DID. The identity certificate issuance module 112 may store, in the first ID registry 610, a DID of an individual to whom a certificate is issued and the fact the issuing of the certificate. The medical institution certificate issuance module 114 may store, in the first ID registry 610, a DID of a target to which a certificate has been issued and the fact of the issuing of the certificate.

In one embodiment, the vaccination management server 200 may include a processor 210, a memory 220, and a communication circuit 230. The processor 210 may control the overall operation of the vaccination management server 200. The vaccination management server 200 may transmit and receive data to and from the other subjects 100, 300, and 400 of the system 10 through the communication circuit 230.

In one embodiment, the memory 220 may include a vaccine DB 222 including information on vaccines. The vaccine DB 222 may include types of data shown in Table 3. Information on vaccines to be described below may include at least one piece of data included in Table 3. A vaccination certificate may be issued based on the vaccine DB 222. In addition, the vaccine DB 222 may be periodically updated according to new information. As the vaccine DB 222 is updated, actions necessary for the medical institution electronic device 300 or the personal electronic device 400 storing the vaccination certificate may be performed. The actions may include, for example, a notification operation and vaccination certificate update operation.

TABLE 3

| | |
|---|---|
| 1 | Vaccine name |
| 2 | Manufacturer |
| 3 | Vaccine type |
| 4 | Administration method, number of vaccinations |
| 5 | Clinical information (allergies, side effects) |
| 6 | Follow-up observation information |
| 7 | Distribution information (serial number, import |
| | date, distribution method, delivery date to |
| | medical institutions) |

In one embodiment, the memory 220 may include a vaccination agency DB 224 including information on the vaccination agency authorized to perform vaccination. The vaccination agency DB 224 may include types of data shown in Table 4. A plurality of vaccination practitioners may exist for one medical institution. Based on the vaccination agency DB 224, the vaccination management server 200 may determine whether to issue a vaccination certificate.

TABLE 4

| | |
|---|---|
| 1 | Medical institution identification number |
| | (e.g., institution registration number) |
| 2 | Vaccination practitioner's name |
| 3 | Vaccination practitioner phone number |
| 4 | Vaccination practitioner identifier (e.g., DID) |
| 5 | Permission status |
| 6 | Medical institution identifier (e.g., DID) |

In one embodiment, the memory 220 may include a vaccination history DB 226 including information on vaccinations that have been performed. The vaccination history DB 226 may include types of data shown in Table 5.

TABLE 5

| | |
|---|---|
| 1 | Identifier of vaccinated person |
| 2 | Vaccine type |
| 3 | Vaccine serial number |
| 4 | Vaccination medical institution ID |
| 5 | Vaccination information such as vaccination |
| | time, place, and number of vaccinations |
| 6 | Follow-up information such as symptoms |
| | after vaccination |

In one embodiment, the processor 210 may include a vaccination certificate issuance module 212 and a vaccination follow-up management module 214. The processor 210 may drive the vaccination certificate issuance module 212 and the vaccination follow-up management module 214 by executing instructions stored in the memory 220. Operations performed by the vaccination certificate issuance module 212 and the vaccination follow-up management module 214 may be understood as operations performed by the processor 210. Other operations described as being performed by the vaccination management server 200 may be understood as operations performed by the processor 210. The vaccination certificate issuance module 212 may issue a vaccination certificate using the vaccine DB 222, the vaccination agency DB 224, and the vaccination history DB 226 stored in the memory 220. For example, the vaccination certificate issuance module 212 may generate a vaccination certificate including at least a part of data included in the vaccine DB 222, the vaccination agency DB 2224, and the vaccination history DB 226. The generated vaccination certificate may be digitally signed with an identifier of the vaccination management server 200.

The vaccination follow-up management module 214 may provide follow-up management to the medical institution electronic device 300 and/or the personal electronic device 400 based on based on the vaccine DB 222, the vaccination agency DB 2224, and the vaccination history DB 226 stored in the memory 220. For example, when new clinical information is added to the vaccine DB 222, the vaccination follow-up management module 214 may transmit the added information to the medical institution electronic device 300 and/or personal electronic device 400.

In one embodiment, the vaccination management server 200 may include a wallet for communication between the vaccination management server 200 and the blockchain network 600. The wallet may include an account of the vaccination management server 200 on the blockchain network 600. For example, the vaccination management server 200 may store a private key in the memory 220. When the vaccination management server 200 issues a certificate, signing may be performed with its own private key.

In various embodiments, the wallet may include a DID agent. The DID agent may receive a DID on the blockchain network 600, retrieve the DID, and change the DID. The vaccination certificate issuance module 212 may store, in the first ID registry 610, a DID of an individual, a DID of a medical institution, and a fact of the issuing of the certificate included in the vaccination certificate.

FIG. 3 is a pair of block diagrams of the medical institution electronic device 300 and the personal electronic device 400 according to one embodiment.

In one embodiment, the medical institution electronic device 300 may include a processor 310, a memory 320, a communication circuit 330, a camera 340, and a display 350. The processor 310 may control the overall operation of the medical institution electronic device 300. The medical institution electronic device 300 may transmit and receive data to and from the other subjects 100, 200, and 400 of the system 10 through the communication circuit 330.

In one embodiment, the memory 320 may store a first application 322. The medical institution electronic device 300 may execute the first application 322 and output an execution screen of the first application 322 through the display 350.

The first application 322 may be understood as an application for receiving a certification service provided on the system 10. The first application 322 may include functions necessary for using the certification service, such as a function of requesting issuance of a medical institution certificate and a vaccination certificate, a function of recognizing information about a vaccine, and a function of recognizing a vaccinated person. The first application 322 may store an identifier (e.g., a DID) of a medical institution.

In one embodiment, when the first application 322 is installed, the medical institution electronic device 300 may generate a medical institution DID through the wallet of the blockchain network 600 included in the first application 322. The locally generated medical institution DID may be stored in the second ID registry 620 of the blockchain network 600. When the medical institution DID is authenticated by a trusted institution, the medical institution DID may be mapped to the existing medical institution information and stored in the medical institution DB 124 and the vaccination agency DB 224 of the identity authentication server 100 and vaccination management server 200. In addition, when the medical institution DID is stored in the first ID registry 610 by the identity authentication server 100 or the vaccination management server 200, the digital signature of the medical institution is in a verifiable state by any third party.

In one embodiment, the memory 320 may store a medical institution certificate 324, a vaccination agency certificate 326, and a vaccination practitioner list 328. The medical institution certificate 324 may be issued by the identity authentication server 100. The vaccination agency certificate 326 may be issued by the vaccination management server 200. The vaccination practitioner list 328 may be understood as a list of practitioners who may perform vaccination among medical personnel engaged in a medical institution.

In various embodiments, the memory 320 of the medical institution electronic device 300 may include a secure element (SE) (not shown). The medical institution certificate 324 and the vaccination agency certificate 326 may be stored in the secure element. For example, direct communication of the secure element with the external devices 100, 200, and 400 may be limited. The first application 322 may relay data transmission and reception between the secure element and the external devices 100, 200, and 400. In another example, the external devices 100, 200, and 400 may communicate with the secure element through short-range wireless communication through the communication circuit 330. The identifier of the medical institution electronic device 300 may be stored in the secure element.

In various embodiments, the medical institution

electronic device 300 may photograph a vaccine to be vaccinated or a person to be vaccinated through the camera 340. For example, the medical institution electronic device 300 may identify a vaccine and/or an individual vaccinated person through the camera 340.

In one embodiment, the personal electronic device 400 may include a processor 410, a memory 420, a communication circuit 430, a camera 440, and a display 450. The processor 410 may control the overall operation of the personal electronic device 400. The personal electronic device 400 may transmit and receive data to and from the other subjects 100, 200, and 300 of the system 10 through the communication circuit 430.

In one embodiment, the memory 420 may store a second application 422. The personal electronic device 400 may execute the second application 422 and output an execution screen of the second application 422 through the display 450.

The second application 422 may be understood as an application for driving an identity certification function and a vaccination certification function provided to an individual on the system 10.

In one embodiment, the memory 420 may store an identity certificate 424 and a vaccination certificate 426. The identity certificate 424 may be issued by the identity authentication server 100. The vaccination certificate 426 may be issued by the vaccination management server 200.

The identity certificate 424 and the vaccination certificate 426 are stored in the personal electronic device 400 and are not stored in the identity authentication server 100, the vaccination management server 200, and the medical institution electronic device 300.

Once the owner of the personal electronic device 400 has received the issued certificate, the owner may independently prove his or her own identity or status of vaccination by personally using a certificate stored in the electronic device 400 without communication with other trusted institution. In addition, the personal electronic device 400 may generate a certificate including only necessary information and share the certificate with a third party, by issuing a processed certificate. In this way, individual sovereignty over data may be strengthened.

In various embodiments, the memory 420 of the personal electronic device 400 may include a secure element (SE) (not shown). The identity certificate 424 and the vaccination certificate 426 may be stored in the secure element. For example, direct communication of the secure element with the external devices 100, 200, and 300 may be limited. The second application 422 may relay data transmission and reception between the secure element and the external devices 100, 200, and 300. In another example, the external devices 100, 200, and 300 may communicate with the secure element through short-range wireless communication through the communication circuit 430.

In one embodiment, the personal electronic device 400 may share a stored certificate or a processed certificate with a third party through the communication circuit 430.

In one embodiment, the memory 420 may store the second application 422. The personal electronic device 400 may execute the second application 422 and output the execution screen of the second application 422 through the display 450.

The second application 422 may be understood as an application for receiving a certification service provided on the system 10. The second application 422 may include functions necessary for using the certification service, such as a function of requesting issuance of an identity certificate and a vaccination certificate and a function of sharing a certificate store in the personal electronic device 400. The second application 422 may store an identifier of an individual (e.g., a DID).

In one embodiment, when the second application 422 is installed, the personal electronic device 400 may generate a user DID through the wallet of the blockchain network 600 included in the second application 422. The locally generated user DID may be stored in the second ID registry 620 of the blockchain network 600. When the user DID is authenticated by the trusted institution, the user DID may be mapped to the existing user information and stored in the personal identity DB 122 and the vaccination history DB 226 of the identity authentication server 100 and vaccination management server 200. In addition, when the user DID is stored in the first ID registry 610 by the identity authentication server 100 or the vaccination management server 200, the digital signature of the user is in a verifiable state by any third party.

In various embodiments, the first application 322 and the second application 422 may include a DID agent. The first application 322 and the second application 422 may receive a DID on the blockchain network 600, retrieve the DID, and change the DID.

Hereinafter, a case in which a certificate issued on the system 10 is a verifiable credential (hereinafter, VC) according to the W3C standard protocol will be described as an example. In addition, the case where a certificate generated by being processed from the VC is a verifiable presentation (hereinafter referred to as VP) designed according to the W3C standard protocol will be described as an example.

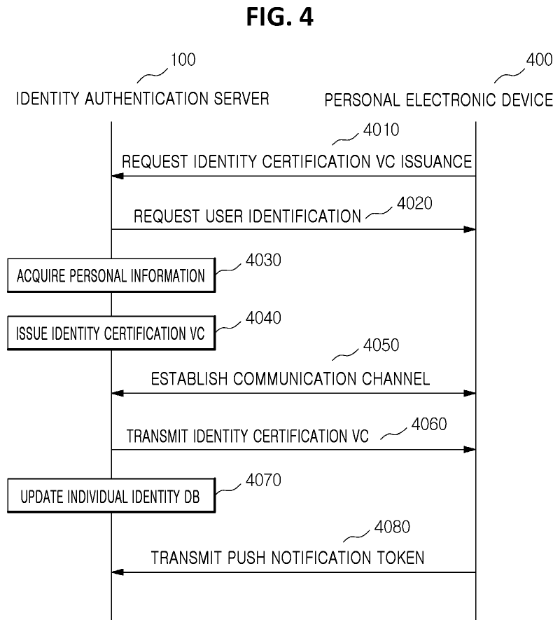

FIG. 4 is a signal flow diagram for a method in which the identity authentication server 100 issues an identity certificate to the personal electronic device 400 according to one embodiment.

The personal electronic device 400 may request the identity authentication server 100 to issue an identity certification VC (the identity certificate described above) (4010). The personal electronic device 400 may drive the second application 422 and transmit the request to the identity authentication server 100 through the second application 422. In operation 4010, the personal electronic device 300 may transmit its own DID (hereinafter referred to as personal DID). The identity authentication server 100 may check the received DID through the personal identity DB 122. The identity authentication server 100 may transmit a user identification request to the personal electronic device 300 (4020). The user identification request may be performed to identify whether the owner of the personal electronic device 300 is a specific individual to whom the identity certification VC is to be issued before the identity certification VC is issued.

The identity authentication server 100 may acquire personal information (4030). For example, the identity authentication server 100 may acquire personal information about the owner of the personal electronic device 300 from a third-party service that provides a user certification service. The personal information may include name, gender, date of birth, mobile phone number, nationality, and personal identification number (e.g., data in Table 1). The identity authentication server 100 may compare the acquired personal information with data stored in the personal identity DB 122 and check whether the two match. In this way, the identity authentication server 100 may check whether an identity certification VC issuance request has been performed by a legitimate user.

The identity authentication server 100 may issue the identity certification VC (4040). The identity certification VC may include a personal DID and personal information received from the personal electronic device 400. The identity certification VC may be digitally signed with an identifier of the identity authentication server 100. The identifier may be, for example, a secret key (DID) of an account of the identity authentication server 100 on the blockchain network 600.

The identity authentication server 100 and the personal electronic device 400 may establish a communication channel for mutual data transmission (4050). The identity authentication server 100 may transmit the identity certification VC to the personal electronic device 400 through the communication channel. The personal electronic device 400 may store the received identity certification VC in the memory 420. The identity certification VC may be referred to as the identity certificate 424 of FIG. 3.

In one example, the communication channel may be an encrypted secret communication channel between DIDs. For example, the communication channel may be a DIDComm communication channel. Hereinafter, the communication channel established to distribute VC may be understood as an encrypted secret communication channel between DIDs. The identity certification VC may be transmitted to the second application 422 of the personal electronic device 400 possessing a DID to which the VC is to be issued through the DIDComm communication channel.

The identity authentication server 100 may update the personal identity DB 122 for the user to whom the identity certification VC has been issued (4070). For example, the identity authentication server 100 may store the DID acquired in operation 4010 in the personal identity DB 122. Accordingly, the DID and the personal information may be stored by being mapped to each other. In addition, in the personal identity DB 122, a record may be made as to whether or not the identity certification VC has been issued.

The identity authentication server 100 may receive and store the push notification token from the second application 422 of the personal electronic device 400 (4080). The identity authentication server 100 may send a push notification to the personal electronic device 400 through the push notification token and the second application 422.

In various embodiments, identity authentication server 100 may record, in the first ID registry 610 of the blockchain network fact 600, the that the identity certification VC for the user DID has been issued. Therefore, the fact that the identity certification VC has been issued for the corresponding DID by the identity certificate authority may be verified.

The method of issuing the identity certificate of FIG. 4 may be similarly applied to the method of issuing a medical institution certificate to the medical institution electronic device 300.

The medical institution electronic device 300 may request a medical institution certification VC (the aforementioned medical institution certificate) issuance request (e.g., operation 4010). The medical institution electronic device 300 may drive the first application 322 and transmit the request to the identity authentication server 100 through the second application 422. In this case, the medical institution electronic device 300 may transmit basic information about the medical institution and the DID of the medical institution stored in the first application 322 to the identity authentication server 100.

The identity authentication server 100 may compare information stored in the medical institution DB 124 based on the received basic information. In this way, the identity authentication server 100 may check whether a medical institution certification VC issuance request has been performed by a legitimate user. When the information stored in the medical institution DB 124 and the received basic information match, the identity authentication server 100 may issue a medical institution certification VC including an identifier of the medical institution and the identifier of the identity certificate authority.

The identity authentication server 100 and the medical institution electronic device 300 may establish a communication channel for mutual data transmission (e.g., operation 4050). The identity authentication server 100 may transmit the medical institution certification VC to the medical institution electronic device 300 through the communication channel. The medical institution electronic device 200 may store the received medical institution certification VC in the memory 320. The medical institution certification VC may be referred to as the medical institution certificate 324 of FIG. 3. The identity authentication server 100 may store the medical institution DB 124 by mapping basic information about the medical institution to which the medical institution certification VC has been issued and the medical institution DID to the medical institution DB 124 (e.g., operation 4070). In addition, a record may be made as to whether or not the medical institution certification VC has been issued in the medical institution DB 124.

In various embodiments, the identity authentication server 100 may record, in the first ID registry 610 of the blockchain network 600, the fact that the medical institution certification VC for the medical institution DID has been issued. Therefore, the fact that the medical institution certification VC has been issued for the corresponding DID by the identity certificate authority may be verified.

Operations 4080 to 4090 of FIG. 4 may be identically performed through the identity authentication server 100 and the first application 322 of the medical institution electronic device 300. In this way, the identity authentication server 100 may transmit a push notification to the medical institution electronic device 300 through the first application 322.

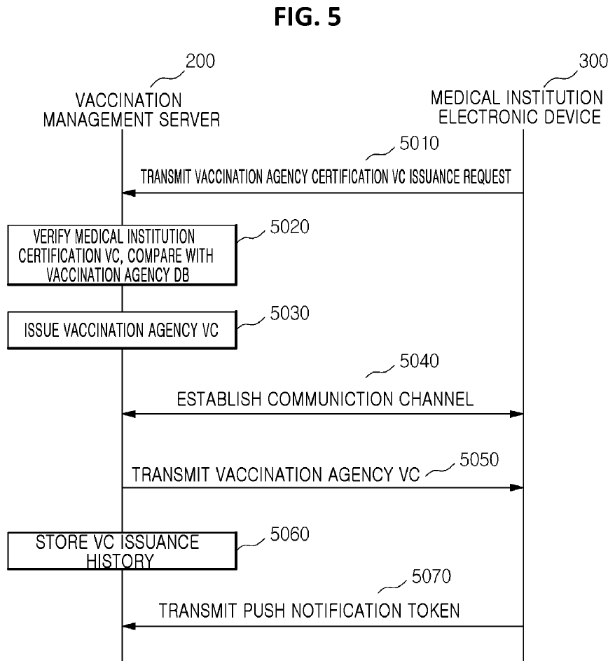

FIG. 5 is a signal flow diagram for a method in which the vaccination management server 200 issues a vaccination agency certificate to the medical institution electronic device 300 according to one embodiment. A vaccination agency may be understood as a medical institution licensed to perform vaccination. A list of vaccination agencies may be designated in advance and stored in the vaccination agency (DB) 224 of the vaccination management server 200.

The medical institution electronic device 300 may transmit a vaccination agency certification VC issuance request to the vaccination management server 200 (5010). The request may include a medical institution certification VC (e.g., the medical institution certificate 324 of FIG. 3) including a digital signature of a vaccination management agency and a digital signature of a medical institution. In various embodiments, the medical institution certification VC may be signed with a medical institution DID stored in the medical institution electronic device 300. The medical institution certification VC digitally signed by the medical institution may have specifications of VP.

The vaccination management server 200 may verify the received medical institution certification VC (5020). The vaccination management server 200 may verify a digital signature of the identity authentication server 100 included in the medical institution certification VC. The above-described verification of the digital signature of the VC issuer may be performed. For example, the vaccination management server 200 may verify a signature of an identity certificate authority included in the medical institution certification VC based on a DID of the trusted institution registered in the first ID registry 610 of the blockchain network 600. For example, the vaccination management server 200 may check whether the DID of the identity certificate authority stored in the first ID registry 610 and the DID derived from the signature of the identity certificate authority match. If the two do not match, the vaccination management server 200 may reject the request in operation 5010 because the medical institution certification VC is not issued by a legitimate identity certificate authority.

The vaccination management server 200 may verify the digital signature of the medical institution. For example, the vaccination management server 200 may check whether the identifier derived from the digital signature and the medical institution identifier pre-stored in the medical institution certification VC match, thereby checking whether the medical a institution certification VC has been transmitted from person having a legitimate authority to possess the VC. If another medical institution makes a lawful request for issuance of the vaccination agency certification VC with the above medical institution certification VC, in operation 5010, the vaccination management server 200 may reject the VC issuance request because the received medical institution certification VC does not include the signature by the medical institution DID stored in the VC.

In various embodiments, the medical institution DID may be replaced with a DID of a representative individual of the medical institution. In this case, an identity certification VC for the representative individual may be treated as the medical institution certification VC.

The vaccination management server 200 may compare information in the vaccination agency DB 224 with information institution about the medical included in the medical institution certification VC (5020). When there is information that matches the information about the medical institution included in the medical institution certification VC in the vaccination agency DB 224, the vaccination management server 200 may confirm that the medical institution that applied for the VC is a medical institution designated as a vaccination agency in advance. The vaccination management server 200 may issue a vaccination agency VC (5030). The vaccination agency VC may include a digital signature of the vaccination agency and an identifier of the medical institution.

The vaccination management server 200 and the medical institution electronic device 300 establish may a communication channel for mutual data transmission (e.g., operation 5040). The vaccination management server 200 may transmit the vaccination agency VC to the medical institution electronic device 300 through the communication channel. The medical institution electronic device 300 may store the received vaccination agency VC in the memory 320. The vaccination agency VC may be referred to as the vaccination agency certificate 326 of FIG. 3.

In one example, the communication channel of operation 5040 may be an encrypted communication channel. For example, the communication channel may be a DIDComm communication channel. The vaccination agency VC may be transmitted to the first application 322 of the medical institution electronic device 300 possessing a DID to which the VC is to be issued through the DIDComm communication channel.

The vaccination management server 200 may store a VC issuance history in the vaccination agency DB (5060). In various embodiments, the vaccination management server 200 may record, in the first ID registry 610 of the blockchain network 600, the fact that the vaccination agency certification VC for the medical institution identifier has been issued. Therefore, the fact that the vaccination agency certification VC has been issued to the corresponding identifier by the vaccination management agency may be verified through the blockchain network 600.

The vaccination management server 200 may receive and store a push notification token from the first application 322 of the medical institution electronic device 300 (5070). The vaccination management server 200 may send a push notification to the medical institution electronic device 300 through the push notification token and the first application 322.

In various embodiments, the medical institution certification VC may be replaced with an identity certification VC of a representative individual of the medical institution. Information on the representative individual may be stored in the vaccination agency DB 224 of the vaccination management server 200 (e.g., Table 2). The representative individual may submit an identity certification VC issued for his/her own identifier (DID) and receive the vaccination agency VC.

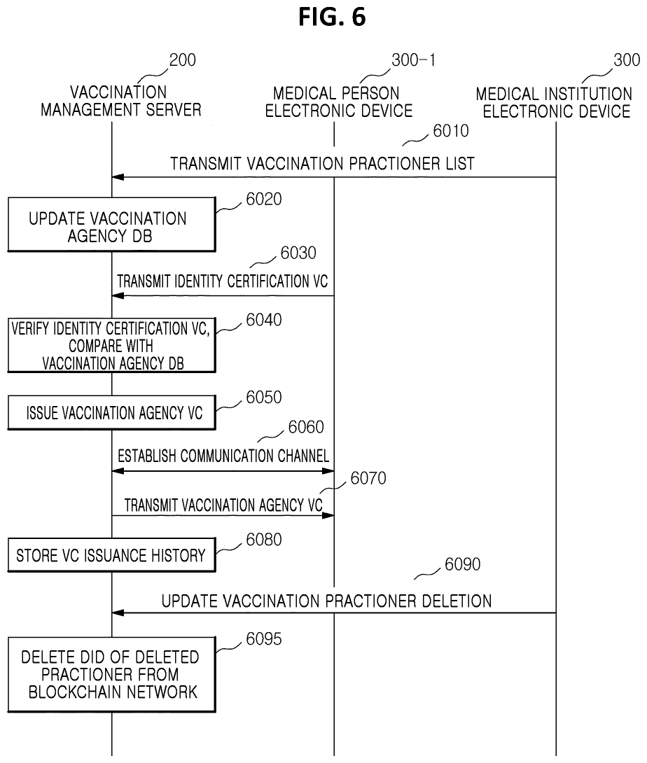

FIG. 6 is a signal flow diagram for a method of registering a vaccination practitioner in the vaccination management server 200 according to one embodiment. The medical institution electronic device 300 may manage a list of practitioners who will perform vaccination among medical persons of the corresponding medical institution. The medical institution electronic device 300 may store information on the vaccination practitioners in the vaccination practitioner list 328. When changes occur in practitioners, the medical institution electronic device 300 may immediately update the change information to the vaccination practitioner list 328.

The medical institution electronic device 300 may transmit the vaccination practitioner list to the vaccination management server 200 (6010). The vaccination management server 200 may update the vaccination agency DB 224. The vaccination practitioner list may be stored by being mapped to medical institutions. The vaccination practitioner list may include identifier information associated with each practitioner.

A medical person who is a vaccination practitioner may download the first application 322 to an electronic device 300-1 possessed by the medical person and generate a DID (hereinafter referred to as a medical person electronic device 300-1). The medical person electronic device 300-1 may register the locally generated DID in the second ID registry 620 of the blockchain network 600. The medical person electronic device 300-1 may receive an identity certification VC for the medical person from the identity authentication server 100. The medical person electronic device 300-1 may have the same block diagram as the medical institution electronic device 300 of FIG. 3.

The medical person electronic device 300-1 may transmit a vaccination agency VC issuance request to the vaccination management server 200 (6030). The request may include an identity certification VC for the medical person and a digital signature of the medical person. The vaccination management server 200 may verify a digital signature of the identity authentication server 100 included in the identity certification VC and the digital signature of the medical person (6040). The vaccination management server 200 may compare practitioner information in the vaccination agency DB 224 with the identity information about the medical person included in the identity certification VC (6040). When there is information matching the identity certification VC in the vaccination agency DB 224, the vaccination management server 200 may confirm that the medical person who has applied for the VC is a medical person who has been certified as the vaccination practitioner in advance. The vaccination management server 200 may issue a vaccination agency VC to a specific medical person (6050). In this case, the vaccination agency VC may include a digital signature of the vaccination agency, an identifier of a medical institution, and an identifier of a medical person.

The vaccination management server 200 and the medical person electronic device 300-1 may establish a communication channel for mutual data communication (6060). The vaccination management server 200 may transmit the vaccination agency VC to the medical person electronic device 300-1 through the communication channel. The medical person electronic device 300-1 may store the received vaccination agency VC in a memory (e.g., the memory 320 of FIG. 3). The vaccination agency VC may be referred to as the vaccination agency certificate 326 of FIG. 3.

The vaccination management server 200 may store a VC issuance history in the vaccination agency DB 224 (6080). In various embodiments, the vaccination management server 200 may record, in the first ID registry 610 of the blockchain network 600, the fact that the vaccination agency VC for a DID of the medical person has been issued. Therefore, the fact that the vaccination agency VC has been issued for the corresponding DID by the vaccination management agency may be verified.

According to various embodiments, when a specific practitioner is deleted from the vaccination practitioner list, the medical institution electronic device 300 may transmit, to the vaccination management server 200, a vaccination practitioner deletion update request (6090). The deletion update request may include identifier information for a practitioner to be deleted.

The vaccination management server 200 may delete the practitioner from the vaccination agency DB 224 and cause a DID of the deleted practitioner to be deleted from the first ID registry 610 of the blockchain network 600 (6080). Then, the vaccination agency VC issued for the DID of the deleted practitioner will be usable.

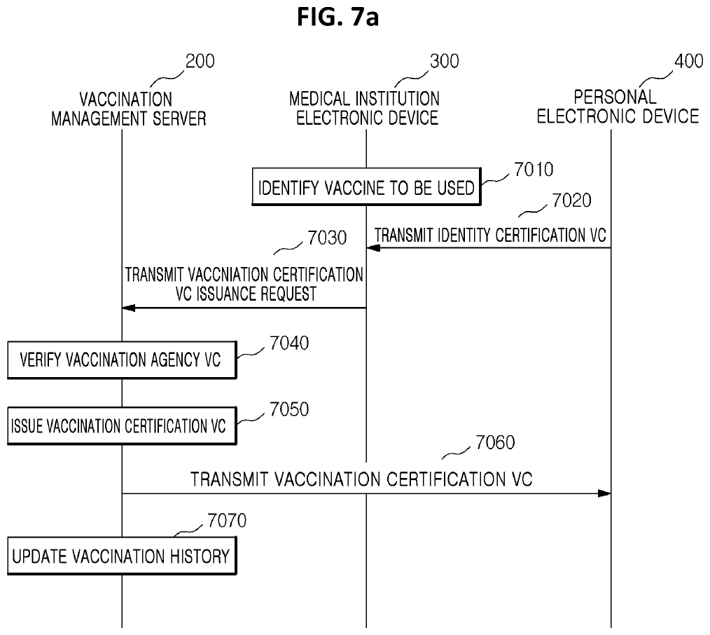

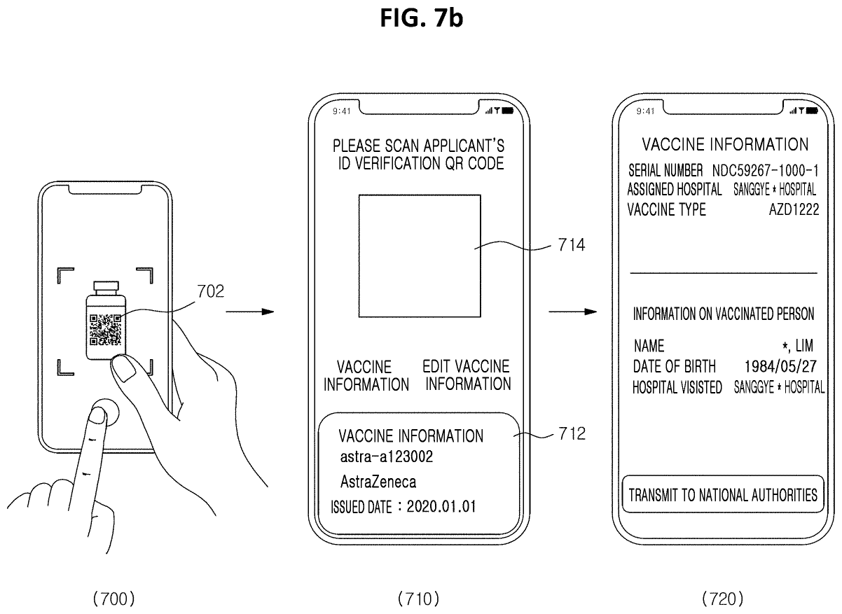

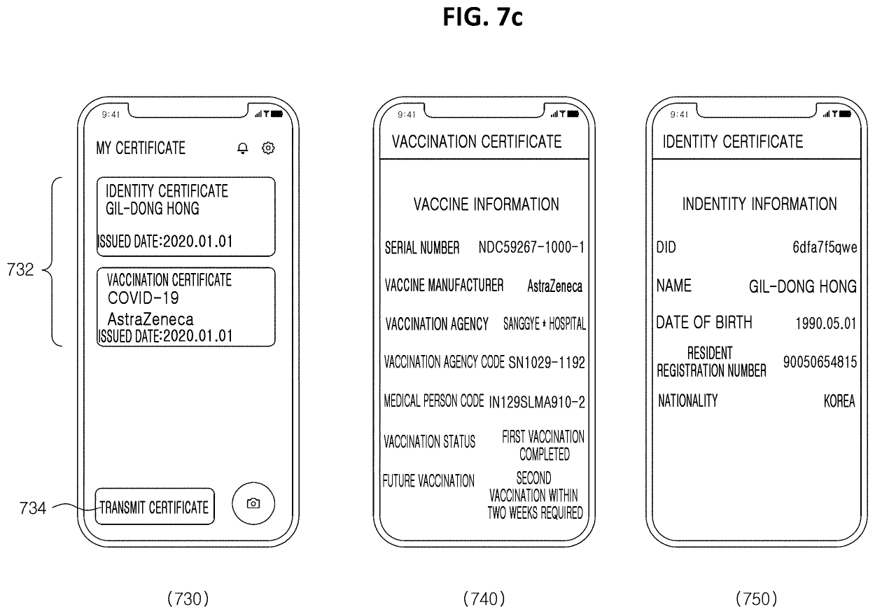

In various embodiments, when a vaccination certificate issuance request is received from a practitioner, in a process of verifying a digital signature of the practitioner, the vaccination management server 200 may check whether an identifier of the practitioner is stored as valid data in the first ID registry 610, in addition to an operation of checking the identifier of the practitioner included in the vaccination agency VC. If the identifier is an identifier deleted from the first ID registry 610 or an identifier stored as unauthorized, the vaccination certificate issuance request by the practitioner may be rejected. Accordingly, in this case, issuance of the vaccination certificate by the medical person electronic device 300-1 associated with the identifier of the deleted practitioner becomes impossible. FIG. 7a is a signal flow diagram for a method of issuing a vaccination certificate according to one embodiment.

In various embodiments, the medical institution electronic device 300 and the medical person electronic device 300-1 may be subjects capable of requesting issuance of a vaccination certificate after vaccination. FIG. 7a illustrates an example in which the medical institution electronic device 300 performs a vaccination certificate issuance request, but the same procedure may be applied to the medical person electronic device 300-1.

The medical institution electronic device 300 may identify a vaccine to be used (7010). For example, the medical institution electronic device 300 may drive the first application 322 and identify a vaccine to be vaccinated. Here, the vaccine to be identified may be a vaccine to be directly vaccinated to a user (a person to be vaccinated). The medical institution electronic device 300 may acquire information about a vaccine to be used, such as a vaccine type, serial number, and distribution information about the corresponding vaccine (e.g., data in Table 3). An identification procedure may be performed by manual input or performed by barcode/QR code recognition.

The medical institution electronic device 300 may receive an identity certification VC from the personal electronic device 400 possessed by the user (7020). The identity certification VC may include the user's digital signature. The medical institution electronic device 300 may verify the digital signature and the identity certification VC, and may identify an individual user through the verification operation.

When the vaccination is completed, the medical institution electronic device 300 may transmit a vaccination certification VC issuance request to the vaccination management server 200 (7030). The vaccination certification VC issuance request may include vaccination information, identity certification VC of the user, the user's digital signature, vaccination agency certification VC of the medical institution, and the medical institution's digital signature. In this case, the vaccine identification information, the identity certification VC, and the vaccination agency certification VC may be digitally signed with the medical institution DID of the medical institution electronic device 300.

The vaccination information may include medical information that needs to be recorded in connection with vaccination, such as identified vaccine information, vaccination time, and vaccination place.

The vaccination management server 200 may verify a vaccination agency VC (7040). The vaccination management server 200 may verify the digital signature of the medical institution included in the request. In this way, it may be verified whether the request of operation 7030 is a request made by a legitimate institution having a vaccination authority.

In various embodiments, the vaccination management server 200 may verify vaccine information included in the request. The vaccination management server 200 may check whether correct vaccine information distributed to the corresponding medical institution has been input by comparing the received vaccine information with the vaccine DB 222. For example, a check as to whether the vaccine matches distribution information in the vaccine DB 222 may be performed. The vaccination management server 200 may check whether the vaccine has been delivered to the corresponding medical institution, whether the vaccine is a licensed medicine, and the like. When it is determined that supplementation is necessary for the corresponding information, the vaccination management server 200 may transmit a supplementation request to the medical institution electronic device 300.

When the verification of the received request is completed, the vaccination management server 200 may issue the vaccination certification VC (7050). The vaccination certification VC may include a user identifier of a vaccinated person, a medical institution identifier, and a digital signature of a vaccination management agency. The vaccination management server 200 may issue the vaccination certification VC by signing the vaccination certification VC with an identifier of the vaccination agency. The vaccination management server 200 may transmit the issued vaccination certification VC to the personal electronic device 400 (7060). The personal electronic device 400 may store the received vaccination certification VC. The vaccination certification VC may be referred to as the vaccination certificate 426.

The user of the personal electronic device 400 may check detailed information about the inoculated vaccine through the second application 422. The vaccination certificate 426 may include the vaccine type, the vaccination date, the vaccination time, information about the medical institution that has performed the vaccination, and information about a medical person who has performed the vaccination.

The vaccination management server 200 may update the corresponding vaccination history to the vaccination history DB 226. For example, the vaccination management server 200 may record, in the vaccination history DB 226, the fact that for the user identifier, vaccination has been performed with a vaccine with a specific identification number by the medical institution identifier and/or the medical person identifier. In various embodiments, the vaccination management server 200 may record, in the first ID registry 610, the fact that the vaccination certification VC has been issued for the user DID.

In various embodiments, the personal electronic device 400 may transmit the vaccination certification VC issuance request in operation 7030 to the vaccination management server 200. For example, the medical institution electronic device 300 may transmit vaccination information to the vaccination management server 200 when vaccination is completed. The personal electronic device 400 may transmit the user's identity certification VC to the vaccination management server 200. The identity certification VC may include the user's digital signature. The vaccination management server 200 may verify the digital signature and the identity certification VC, and may identify the individual user through the verification operation. In this case, the vaccination management server 200 may issue the vaccination certification VC based on the vaccination information and the identity certification VC received from the medical institution electronic device 300 (e.g., operation 7050).

FIG. 7b is an example of a screen interface displayed on the medical institution electronic device 300 according to one embodiment. As the first application 322 is driven, the medical institution electronic device 300 may output a screen 700 to a screen 720 through the display 350.

In operation 7010 of FIG. 7a according to one embodiment, the medical institution electronic device 300 may recognize a QR code 702 of the vaccine. The QR code 702 of the vaccine may be affixed to or printed on a bottle of the vaccine. The QR code 702 of the vaccine may include information on the vaccine to be used for vaccination. For example, the information on the vaccine may include a serial number, an import date, a distribution method, and a delivery date to a medical institution. The information on the vaccine may correspond to distribution information (Table 3) of the vaccine DB 222 of the vaccination management server 200. In various embodiments, in addition to the QR code, other types of codes, such as barcodes, may be affixed to the vaccine.

Referring to the screen 700, the medical institution electronic device 300 may drive the camera 340 and recognize the QR code 702 of the vaccine. The medical institution electronic device 300 may acquire information on the vaccine by recognizing the QR code 702.

In another embodiment, the medical institution electronic device 300 may acquire the information on the vaccine through an input device (e.g., a touch screen or a keyboard). In this case, the medical institution electronic device 300 may display an item to be input through the screen and receive a data value corresponding to the item.

In various embodiments, the identity certification VC may be converted into a QR code and output through the display 450 of the personal electronic device 400. The medical institution electronic device 300 may acquire the VC by recognizing the displayed QR code. The medical institution electronic device 300 performs verification on the acquired VC. By utilizing QR code recognition, direct exposure of information may be minimized, especially when identity certification is performed by private units.

In operation 7020 of FIG. 7a according to one embodiment, the medical institution electronic device 300 may acquire the identity certification VC by recognizing the QR code displayed through the display 450 of the personal electronic device 400. Referring to the screen 710, the medical institution electronic device 300 may output information 712 on the recognized vaccine through the display 350, and may output a guide 714 for acquiring the user's identity certification VC.

In operation 7030 of FIG. 7a according to one embodiment, the medical institution electronic device 300 may display the recognized vaccine information and the received identity information through the screen 720. A vaccination practitioner possessing the medical institution electronic device 300 may check the input information through the screen 720 and transmit the information to the vaccination management server 200, thereby requesting issuance of a vaccination certificate.

FIG. 7c is an example of a screen interface displayed on the personal electronic device 400 according to one embodiment. As the second application 422 is driven, the personal electronic device 400 may output a screen 730 to a screen 750 through the display 450.

The screen 730 may include a certificate list 732 stored in the personal electronic device 400. The identity certification VC acquired through operation 4060 of FIG. 4 (e.g., the identity certificate 424 of FIG. 3) may be included in the list 732 as an identity certificate. The vaccination certification VC acquired through operation 7060 of FIG. 7a (e.g., the vaccination certificate 426 of FIG. 3) may be included in the list 732 as a vaccination certificate.

The screen 740 is an example of a screen displaying information included in the vaccination certification VC. The screen 750 is an example of a screen displaying information included in the identity certification VC.

In various embodiments, the personal electronic device 400 may transmit at least one of certificates included in the certificate list 732 to an external device when an input to the button 734 is received on the screen 730.

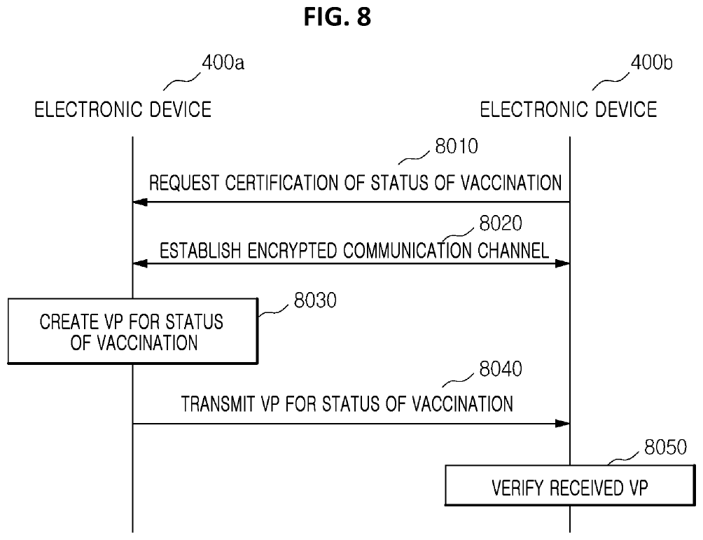

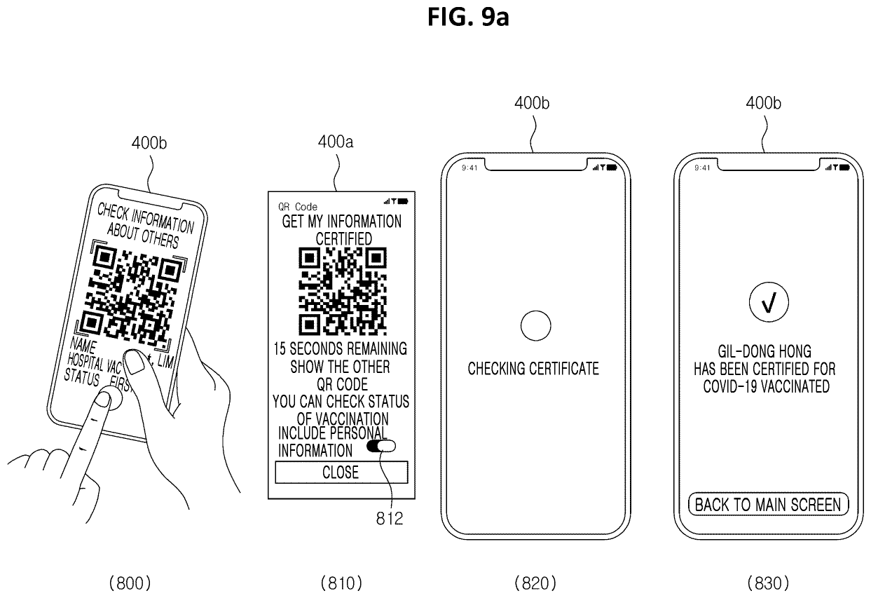

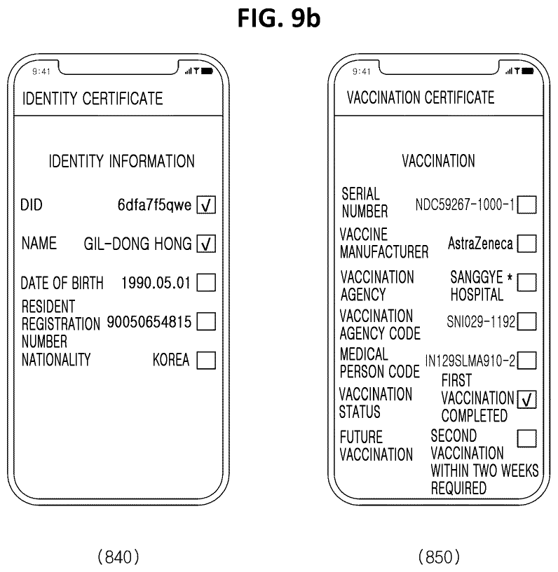

FIG. 8 is a signal flow diagram for a method of certificating a status of vaccination according to one embodiment. FIG. 9a is an example of a screen interface showing a certification process between an A electronic device 400a and a B electronic device 400b according to one embodiment. FIG. 9b is an example of a screen interface for selecting information to be included in a VP to be created in the A electronic device 400a according to one embodiment.

An individual who has completed vaccination possesses the vaccination certificate 426 in the personal electronic device 400. Through the second application 422 of the personal electronic device 400, whether or not vaccination may certificate the status of vaccination for a subject requesting certification on the status of vaccination.

Referring to FIG. 8, an example of certificating the status of vaccination between an individual A and an individual B is illustrated. However, certification of vaccination may be performed by any third party, such as a company or a service. Any subject possessing the personal electronic device 400 in which the second application 422 providing the vaccination certification function is installed may perform the certification. Hereinafter, a description will be given with reference to FIGS. 8, 9a, and 9b.

The A electronic device 400a may receive a certification request for the status of vaccination from the B electronic device 400b (8010). For example, the B electronic device 400b may drive the second application 422 and select the A electronic device 400a to which the certification request is to be transmitted.

In another embodiment, the B electronic device 400b may drive the second application 422 and output a screen displaying the certification request. When the user A and the user B perform mutual certification in a directly contacting state, the A electronic device 400a may output a screen including a code (e.g., the QR code or barcode) corresponding to the aforementioned VP. The B electronic device 400b may receive the VP and certificate the status of vaccination by recognizing the code output on the screen of the A electronic device 400a.

Referring to a screen 800 of FIG. 9b, the B electronic device 400b may output a guide for recognizing the QR code of the personal electronic device 400 to acquire a VP. The A electronic device 400a may output a screen 810 including a QR code corresponding to a VP.

In another example, when the A electronic device 400a and the B electronic device 400b are positioned close to each other by a certain distance or more, the A electronic device 400a and the B electronic device 400b may be configured to automatically request mutual certification. When the certification request is generated, the A electronic device 400a and the B electronic device 400b may establish an encrypted communication channel (8020).

In one embodiment, the A electronic device 400a may

create a VP including only some information included in the vaccination certification VC (e.g., the vaccination certificate 426) (8030). The A electronic device 400a may digitally sign with the user A's identifier for at least some of the information about the vaccination certification VC stored in the A electronic device 400a.

For example, the A electronic device 400a may create a VP including information about the status of vaccination and a digital signature of the user A. The VP may include the digital signature of the vaccination management agency included in the vaccination certification VC, an identifier information about the user A, and the user A's digital signature included in the vaccination certification VC.

The A electronic device 400a may transmit the VP regarding the status of vaccination to the B electronic device 400b (8040). The B electronic device 400b may verify the received VP (8050). Referring to a screen 820 of FIG. 9b, a standby screen while the B electronic device 400b verifies the received certificate is exemplified. The B electronic device 400b may verify that the vaccination certification VC has been issued by a legitimate vaccination management agency based on the identifier of the vaccination management agency stored in the first ID registry 610 of the blockchain network 600 and the digital signature of the vaccination management agency included in the VP. The B electronic device 400b may verify that the vaccination certification VC has been issued to the user A based on the user A's digital signature and identifier information about the user A included in the vaccination certification VC. When the verification is completed, the B electronic device 400b may acquire data on the status of vaccination included in the VP. That is, the individual B will be able to trust the data contained in VP. In this way, exposure of personal information may be minimized by using a zero-knowledge proof that certificates only information on the status of vaccination.

In one embodiment, the user A may select whether to include personal information when creating the VP for the status of vaccination. For example, when a button 812 on the screen 810 is clicked, the VP may be configured to include the status of vaccination and information on the user A′s name. Referring to a screen 830 of FIG. 9b, a screen as a result of certification of “Name-GIL-DONG HONG” in information included in the identity certificate and “STATUS OF VACCINATION” in information included the vaccination certificate is in illustrated as an example.

In various embodiments, the A electronic device 400a may issue a VP including only some selected in information included in the identity certification VC and the vaccination certification VC. Depending on the type of certification request, information included in the VP may be different. When a VP is composed of VCs issued by a plurality of subjects, the VP may include all digital signatures of the plurality of subjects.

Since the system 10 based on the blockchain network 600 is an open system, anyone using the same DID agent may use the certification service provided by the system 10. In addition, certification may be freely performed in the private sector without using a government agency or a specific trusted institution as an intermediary.

In various embodiments, the operations described in FIG. 8 may be configured to certificate various information handled on the certification service of the system 10 in addition to the certification of the status of vaccination. Hereinafter, a description will be given with reference to FIGS. 8, 9a, and 9b.

In operation 8010 according to various embodiments, the B electronic device 400b may transmit a certification request for randomly selected information to the personal electronic device 400. The certification request for the randomly selected information may be a certification request for various information handled in the certification service of the system 10, such as a certification request for the status of vaccination, a certification request for the status of vaccination and identity certification, a certification request for the medical institution where the vaccination has been performed, or the like.

The B electronic device 400b may receive a selection input for information for which certification is to be requested. For example, the second application 422 may provide a list of information provided by trusted institutions on the system 10. The user B may select, from the list, information for which certification is to be requested from the user A. When a selection input corresponding to the selection is received, the B electronic device 400b may transmit a certification request for the information to the A electronic device 400a. Since information provided by the trusted institutions on the system 10 is capable of being verified by the first ID registry 610 of the blockchain network 600, the certification service for randomly selected information may be provided.

Referring to screens 840 and 850 of FIG. 9c, a screen for selecting information to construct a VP is illustrated as an example. The screen 840 is a list of information included in the identity certification VC (identity certificate). An example in which “DID” and “NAME” are selected from the list is illustrated. The screen 850 is a list of information included in the vaccination certification VC (vaccination certificate). An example in which “STATUS OF VACCINATION” is selected from the list is illustrated. Based on the VP including the selected information, “NAME” and “STATUS OF VACCINATION” may be certificated. A certification result screen is illustrated on the screen 830 described above.

In various embodiments, the B electronic device 400b may transmit the certification request using short-range communication or Bluetooth communication with the A electronic device 400a. For example, devices on which the second application 422 installed is may be configured to automatically transmit the certification request to each other when they are positioned closer than a predetermined distance. For example, the B electronic device 400b may transmit the certification request by tagging the A electronic device 400a. The tagging input may be, for example, NFC tagging.

In various embodiments, the B electronic device 400b may transmit the certification request to the personal electronic device 400 through a wired or wireless network. The B electronic device 400b may install the second application 422 or transmit the certification request to the electronic device (e.g., the personal electronic device 400) of the user registered in the certification service provided by the system 10.

For example, the user B may retrieve or search for users registered in the service on the second application 422. The user B may transmit a specific certification request for a retrieved user.

In operation 8030, the A electronic device 400a may select information for constructing a certificate in response to the certification request. For example, information to be selected corresponding to the specific certification request may be designated in advance through the second application 422. Alternatively, the A electronic device 400 may receive a selection input for information for constructing a certificate from the user A. The A electronic device 400a may create a VP including the selected information.

In various embodiments, the personal electronic device 400 may retrieve or search for a third party to which the certificate is to be provided on the second application 422. The personal electronic device 400 may transmit the certificate to the retrieved third party through a wired or wireless network.

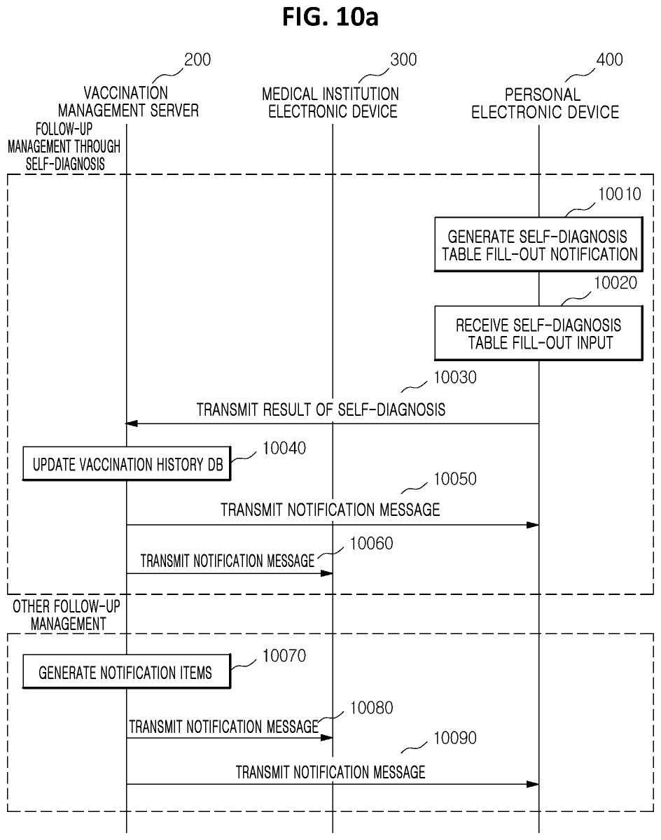

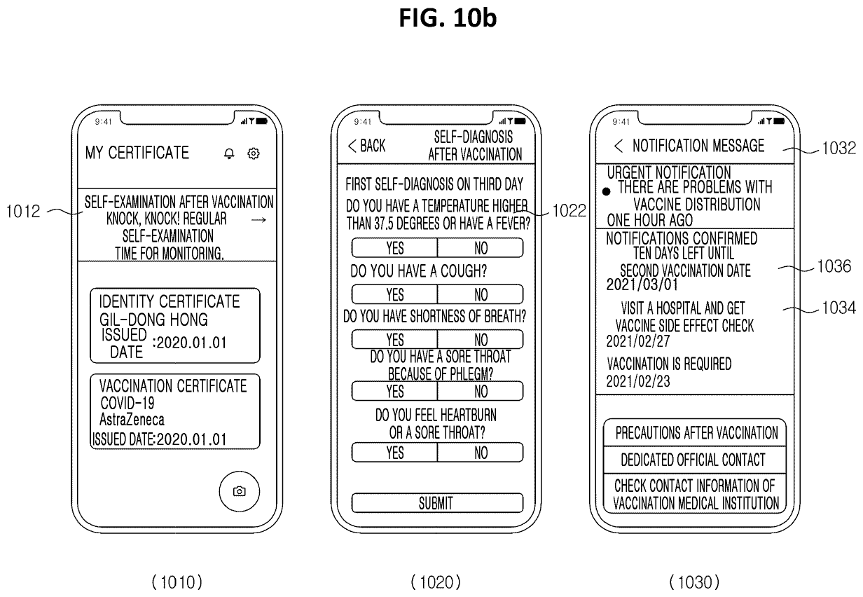

In various embodiments, the system 10 may provide a follow-up management service after vaccination. Referring to FIG. 10a, a signal flow diagram of the follow-up management service according to one embodiment is illustrated. Referring to FIG. 10b, an example of a screen interface in a follow-up management process according to one embodiment is illustrated.

In various embodiments, information included in the vaccine DB 222, the vaccination agency DB 224, and the vaccination history DB 226 of the vaccination management server 200 may be periodically updated. For example, when a new vaccine is developed or a new side effect occurs in an existing vaccine, information about this may be updated in the vaccine DB 222.

For example, when a new medical institution is recognized as a vaccination agency, or when an existing vaccination agency is deprived of vaccination authority, information about this may be updated in the vaccination agency DB 224. For example, information about a vaccinated person, such as when a person, who has been vaccinated with a first vaccine, completes a second vaccination, may be updated in the vaccination history DB 226.

Based on update details of information included in the vaccine DB 222, the vaccination agency DB 224, and the vaccination history DB 226 of the vaccination management server 200, the vaccination management server 200 may change the first ID registry 610 of the blockchain network 600. For example, an identifier corresponding to a medical institution or medical person who has been deprived of the vaccination authority may be deleted from the first ID registry 610.

The personal electronic device 400 on which the second application 422 is installed may receive a push notification from the vaccination management server 200. The vaccination management server 400 may transmit a push notification to the personal electronic device 400 when a preset condition is satisfied.

may periodically generate a self-diagnosis table fill-out notification (10010). For example, the notification may be received from the vaccination management server 200, or the personal electronic device 400 may generate the notification at a preset cycle. For example, the personal electronic device 400 may be configured to, when self-diagnosis is required every week, generate the notification when one week has elapsed since the last self-diagnosis table was input.

Referring to a screen 1010 of FIG. 10b, a self-diagnosis table fill-out notification 1012 is displayed on the screen of the second application 422. When the notification 1012 is clicked, the screen 1010 may be converted to a screen 1020.

The personal electronic device 400 may receive a self-diagnosis table fill-out input from the user (10020). Referring to the screen 1020 of FIG. 10b, a screen displaying a self-diagnosis table 1022 is illustrated as an example. The self-diagnosis table 1022 may be prepared to check vaccine-related side effects or sequela-related symptoms.

In one embodiment, items of the self-diagnosis table 1022 may be received from the vaccination management server 200. The items of the self-diagnosis table 1022 may be prepared to correspond to clinical information and follow-up information (e.g., Table 3) of the vaccine DB 222 of the vaccination management server 200.

When the self-diagnosis table is completed, the personal electronic device 400 may transmit a result of the self-diagnosis to the vaccination management server 200 (10030). The vaccination management server 200 may update the vaccination history DB 226 based on the received result of the self-diagnosis. For example, the vaccination management server 200 may update follow-up information (e.g., Table 5) for an individual have filled out the self-diagnosis table included in the vaccination history DB 226.

When it is necessary to transmit a feedback based on the result of the self-diagnosis, the vaccination management server 200 may transmit the feedback to the personal electronic device 400 or the medical institution electronic device 300 (10050, 10060).

For example, when it is determined that the user needs to visit a hospital as the result of the self-diagnosis, a notification for requesting a visit to the hospital may be transmitted to the personal electronic device 400 as feedback, and a notification for requesting the user to visit the hospital may be transmitted to the medical institution electronic device 300 of the medical institution where the user has been vaccinated as feedback.

In one embodiment, the vaccination management server 200 may generate notification items (10070). The vaccination management server 200 may transmit a notification message to the personal electronic device 400 and/or the medical institution electronic device 300 in response to the generating (10080, 10090).

For example, when new side effect information is updated in the vaccine DB 222, the vaccination management server 200 may transmit a notification message including the new side effect information to the personal electronic device 400. When the user needs to visit the hospital according to the side effect information, the vaccination management server 200 may transmit a notification message to the medical institution electronic device 300.

Referring to a screen 1030 of FIG. 10b, a screen in which a notification message 1032 is displayed is illustrated as an example. A notification 1034 may be a notification message generated corresponding to side effect information updated in the vaccine DB 222.

For example, for a vaccine requiring two vaccinations, when a vaccination certificate for the first vaccination is issued, a notification message 1036 for a second vaccination may be received. If the second vaccination is performed by a scheduled vaccination date, the vaccination certificate for the first vaccine stored in the personal electronic device 400 may be replaced with a certificate including whether or not the second vaccination is performed. For example, the vaccination management server 200 and the medical institution electronic device 300 may re-issue a vaccination certificate for the second vaccination and transmit the re-issued the vaccination certificate to the personal electronic device 400.

If, for the vaccine requiring two vaccinations, the second vaccination is not performed until the scheduled vaccination date, the vaccination management server 200 may invalidate the previously issued vaccination certificate. The vaccination certificate may have a unique identifier, and the unique identifier may be a hash value for all contents of the vaccination certificate. When the certificate is invalidated, information indicating the fact that the certificate is invalid may be recorded in the blockchain network 600 for the unique identifier of the certificate.

In various embodiments, the first ID registry 610 may store a unique identifier value for the VC (certificate) issued by a trusted institution. The recipient of the certificate may perform certificate verification based on the signature of the issuer included in the certificate and the unique identifier value derived from the certificate. For example, when the unique identifier value calculated from the received certificate is not the value stored in the first ID registry 610, it may be determined that the received certificate is invalid or a forged certificate after being issued.

In various embodiments, the vaccination management server 200 may invalidate the previously issued certificate according to updated information. Therefore, since certification of the certificates distributed on the system 10 may be conveniently performed between individuals while information is managed by the trusted institution, reliability of certificates may be increased.

Electronic devices according to various embodiments disclosed in this document may be devices of various types. The electronic devices may include, for example, a portable communication device (e.g. smartphone), a computer device, a portable multimedia device, a portable medical device, a camera, a wearable device, or a home appliance. The electronic device according to an embodiment of the present document is not limited to the aforementioned devices.

Various embodiments of this document and terms used therein are not intended to limit the technical features described in this document to specific embodiments, and should be understood to include various modifications, equivalents, or alternatives of the embodiments. In relation to the description of the drawings, similar reference numerals may be used for similar or related components. The singular form of a noun corresponding to an item may include one item or a plurality of items, unless the relevant context clearly dictates otherwise. In this document, each of phrases such as “A or B”, “at least one of A and B”, “at least one of A or B”, “A, B or C”, “at least one of A, B and C”, and “at least one of A, B, or C” may include all possible combinations of items listed together in the corresponding phrase among those phrases. Terms such as “first”, “second”, “firstly”, or “secondly” may simply be used to distinguish a corresponding component from other corresponding components, and do not limit the corresponding components in other respects (e.g., importance or order). In this document, if a certain (e.g., first) element is referred to as being “connected” or “coupled” with or without the terms “functionally” or “communicatively” to another (e. g., second) component, it means that the certain component can be connected to the other component directly (e.g., in a wired manner), wirelessly, or through a third component.

Various embodiments as set forth herein may be implemented as software (e.g., the program 422) including one or more instructions that are stored in a storage medium (e.g., the internal memory 420 or external memory 420) that is readable by a machine (e.g., the electronic device 400). For example, a processor (e.g., the processor 410) of the machine (e.g., the electronic device #01) may invoke at least one of the one or more instructions stored in the storage medium, and execute it. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments disclosed in this document may be provided by being included in a computer program product. The computer program product may be traded between a seller and a buyer as a commodity. The computer program product may be distributed in the form of a machine-readable storage medium (e.g., compact disc read only memory (CD-ROM)), or may be distributed (e.g., downloaded or uploaded) online, through an application store (e.g., Play Store™) or directly between two user In case of online devices (e.g., smartphones). the distribution, at least part of the computer program product may be temporarily stored or tentatively generated in the machine-readable storage medium such as a memory of a manufacturer's server, application store server, or relay server.

According to various embodiments, each component (e.g., module or program) of the components described above may include one or a plurality of entities. According to various embodiments, one or more components among the components described above or operations may be omitted, or one or more other components or operations may be added. Alternatively or additionally, a plurality of components (e.g., modules or programs) may be integrated into one component. In this case, the integrated component may perform one or more functions of each component of the plurality of components identically or similarly to those functions performed by a corresponding component among the plurality of components prior to the integration. According to various embodiments, operations performed by modules, programs, or other components may be executed sequentially, in parallel, iteratively, or heuristically, or one or more of the operations may be executed in a different order or omitted, or, one or more other operations may be added.

1. A method for vaccination management including a trusted institution server associated a trusted with institution, a medical institution device associated with a medical institution, a user device associated with a user, and a blockchain network that includes a public distributed ledger,
a first storage space of the public distributed ledger being set to be modifiable only by the trusted institution server, an identifier of the trusted institution and an identifier of the medical institution being stored in the first storage space,
the method comprising:
transmitting, by the medical institution device, a vaccination certificate issuance request including vaccination information, an identity certification verifiable credential (VC) for the user received from the user device, a vaccination agency VC for the medical institution, and a digital signature of the medical institution, to the trusted institution server;
verifying, by the trusted server, institution the vaccination agency VC based on a digital signature of a VC issuer included in the vaccination agency VC and the identifier of the trusted institution stored in the blockchain network, in response to the vaccination certificate issuance request;
determining, by the trusted institution server, whether or not there is an authority for the vaccination certificate issuance request based on the vaccination agency VC and a medical institution database stored in the trusted institution server, in response to the vaccination certificate issuance request;
issuing, by the trusted institution server, a vaccination certification VC including the vaccination information, at least some data included in the identity certification VC, and a digital signature of the trusted institution, based on the verification result and the determination result; and
transmitting, by the trusted institution server, the vaccination certification VC to the user device.

2. The method of claim 1, wherein the verifying of the vaccination agency VC includes comparing an identifier calculated from the digital signature of the VC issuer with the identifier of the trusted institution.

3. The method of claim 1, wherein the determining of whether or not there is the authority for the vaccination certificate issuance request includes checking whether the medical institution stored by being mapped to an identifier calculated from a digital signature of an owner of the vaccination agency VC included in the vaccination agency VC is a certified vaccination agency.

4. The method of claim 1, further comprising generating a certificate identifier corresponding to the vaccination certification VC generated by the trusted institution server and transmitting the certificate identifier to the blockchain network so that the certificate identifier is stored in the first storage space.

5. The method of claim 4, further comprising transmitting, by the trusted institution server, a request to delete the certificate identifier from the first storage space to the blockchain network, when the vaccination certification VC expires.

6. The method of claim 1, further comprising:
transmitting, by the user device, the vaccination certification VC and a digital signature of the user to a third party device;
verifying, by the third party device, a vaccination VC based on a digital signature of an issuer of the vaccination certification VC included in the vaccination certification VC and the identifier of the trusted institution stored in the blockchain network; and
verifying, by the third party device, an owner of the vaccination VC based on a digital signature of the user and an identifier of an owner of the vaccination certification VC.

7. The method of claim 6, wherein the verifying of the vaccination VC by the third party device includes comparing, by the third party device, an identifier calculated from the digital signature of the issuer of the vaccination certification VC with the identifier of the trusted institution.

8. The method of claim 6, wherein the verifying of the owner of the vaccination VC by the third party device includes comparing, by the third party device, the identifier of the owner of the vaccination VC with the identifier calculated from the digital signature of the user.

9. A system comprising:
a trusted institution server associated with a trusted institution;
a medical institution device associated with a medical institution;
a user device associated with a user; and
a blockchain network that includes a public distributed ledger, a first storage space of the public distributed ledger being set to be modifiable only by the trusted institution server, an identifier of the trusted institution and an identifier of the medical institution being stored in the first storage space,
wherein the medical institution device transmits a vaccination certificate issuance request including vaccination information, an identity certification verifiable credential (VC) for the user received from the user device, a vaccination agency VC for the medical institution, and a digital signature of the medical institution, to the trusted institution server,
the trusted institution server verifies the vaccination agency VC based on a digital signature of a VC issuer included in the vaccination agency VC and the identifier of the trusted institution stored in the blockchain network, in response to the vaccination certificate issuance request,
the trusted institution server determines whether or not there is an authority for the vaccination certificate issuance request based on the vaccination agency VC and a medical institution database stored in the trusted institution server, in response to the vaccination certificate issuance request,
the trusted institution server issues a vaccination certification VC including the vaccination information, at least some data included in the identity certification VC, and a digital signature of the trusted institution, based on the verification result and the determination result, and
the trusted institution server transmits the vaccination certificate to the user device.

10. The system of claim 9, wherein in the verifying of the vaccination agency VC, the trusted institution server verifies the vaccination agency VC by comparing an identifier calculated from the digital signature of the VC issuer with the identifier of the trusted institution.
